(19) United States
(12) Patent Application Publication
(10) Pub. No.: US 2014/0100282 A1
(43) Pub. Date: Apr. 10, 2014

(54) INTRANASAL ADMINISTRATION OF PHARMACEUTICAL AGENTS FOR TREATMENT OF NEUROLOGICAL DISEASES

(71) Applicant: Patrick S L Wong, Burlingame, CA (US)

(72) Inventor: Patrick S L Wong, Burlingame, CA (US)

(21) Appl. No.: 14/051,344

(22) Filed: Oct. 10, 2013

Related U.S. Application Data
(60) Provisional application No. 61/711,968, filed on Oct. 10, 2012.

(51) Int. Cl. A61K 47/12; A61K 45/06; A61K 31/155; A61K 31/195; A61K 31/135

(52) U.S. Cl. CPC A61K 47/12; A61K 31/195; A61K 31/135; A61K 31/155; A61K 45/06
USPC 514/561; 514/654; 514/635

(57) ABSTRACT

Pharmaceutical formulations for treating neurological diseases are described, wherein the formulations comprise a pharmaceutically active agent-transport moiety complex. The formulations are suitable for administration via an intranasal route. Neurological diseases and conditions are associated with reduced brain insulin signaling (i.e., CNS insulin insensitivity), reduced dopaminergic signaling, reduced serotonergic signaling, reduced cholinergic signaling, or reduced GABAergic signaling, and include Alzheimer's disease, Parkinson's disease, epilepsy, neuropathic pain, fibromyalgia, post-herpetic neuralgia, insomnia, or anxiety. Neurological diseases also include cancers of the central nervous system (CNS).

### CROSS-REFERENCE TO RELATED APPLICATION

This application claims priority from U.S. Provisional Patent Application No. 61/711,968, filed Oct. 10, 2012, which is incorporated by reference herein in its entirety.

### FIELD OF THE INVENTION

This invention relates generally to diagnosis and treatment of patients suffering from neurological disorders including Parkinson's dementia, Alzheimer's disease, central nervous system (CNS) cancers, and the like.

### BACKGROUND OF THE INVENTION

Pharmaceutical agents have been formulated for oral delivery to provide immediate release, sustained release, pulsatile release, duodenal delivery, and the like. A typical objective has been to provide a sustained release formulation capable of providing once daily dosing. One such sustained release approach was to increase the residence time in the upper G.I. tract. For example, PCT Publication WO 1999/047128 describes metformin delivery systems which are predicated on the principle that absorption of metformin occurs primarily only in the upper G.I. tract, and not in the lower G.I. tract. Other prior art attempts to formulate a once-daily dose for metformin were largely unsuccessful. For example, GLUCOPHAGE® XR is proposed as a once-daily formulation, but releases 90% of its dose in about six hours in vitro, too short a time period for a proposed 15 to 20 hour once-daily dosage form. Thus, twice daily dosing of GLUCOPHAGE XR is required. Others have proposed an extended release dosage form of metformin HO, where absorption time in the stomach is extended by increasing the retention time of the dosage form in the stomach (U.S. Pat. No. 6,451,808; U.S. Pat. No. 6,723,340).

In another approach, Wong et al. describe the preparation of a metformin-fatty acid complex for colonic delivery (U.S. Patent Application Publication Nos. 2005/0158374 and 2006/0094782). This formulation was able to provide systemic delivery of metformin for 10 to 24 hours when absorption by colonic delivery, enabling the once daily administration of metformin. A pore size of 2.3 Å has been estimated for colon in comparison to the jejunum (8 Å) and ileum (4 Å) by studying the effects of osmolality on net water movement in the human colon, (Billich et al. 1969 J. Clin. Invest. 48, 1336).

Numerous formulations exist for systemic delivery of pharmaceutical agents by oral administration, providing both immediate and sustained release. However, treatment of neurological diseases such as Alzheimer's disease requires that pharmacological agents gain access to the central nervous system (CNS). The blood-brain barrier (BBB) is formed by the endothelial cells lining the capillaries in the brain, and the pore size between cells in the BBB is likely to be even smaller than that measured in the colon. This barrier represents the strictest barrier for drug therapy and prevents access of pharmacological agents to the CNS. The BBB excludes from the brain ˜100% of large molecules, and more than 98% of all small molecule drugs. (Betz, A. L., et al. 1986 Ann. Rev. Physiol. 48, 841). Radiolabeled histamine (MW˜100 g/mole) was injected intravenously in mouse, and autoradiography performed after 30 minutes showed that there was zero penetration into the brain or spinal cord through the BBB, while the histamine readily crosses the porous capillaries of all peripheral tissues (Pardridge, W. M. 2005 NeuroRx 2, 3-14). Among the small molecules, hydrophilic and charged molecules are particularly impermeable to the BBB.

Intranasal delivery of active agents effectively bypasses the BBB, and can provide an effective route for delivery of active agents to the central nervous system. For example, the following classes of therapeutics have successfully been intranasally delivered to the CNS, though the amounts reaching the brain are small: neurotrophins (NGF and insulin-like growth factor [IGF]-1); neuropeptides (hypocretin-1 and exendin); cytokines (interferon β-1b and erythropoietin); polynucleotides (DNA plasmids and genes); and small molecules (chemotherapeutics and carbamazepine). (See Hansen et al. BMC Neuroscience 2008 9, S5 for review). Intranasal delivery reportedly works best for potent therapeutics that are active in the nanomolar range.

Diabetes is associated with premature mortality and is a risk factor for mild cognitive impairment and both vascular dementia and Alzheimer's disease. It has been suggested that cognitive decline is a consequence of reduced insulin action in the brain. In individuals without diabetes, worse glucoregulation (as measured by a glucose tolerance test) was associated with worse outcomes on cognitive assessment, especially in elderly individuals.

Hyperphosphorylated tau is believed to play an important role in the formation of neurofibrillary tangles in brains of patients with Alzheimer's disease (AD) and related tauopathies and to be a crucial factor in the pathogenesis of these disorders. Though diverse kinases have been implicated in tau phosphorylation, protein phosphatase 2A (PP2A) has been suggested to be the major tau phosphatase. For example, Kickstein, et al. reported that the antidiabetic drug metformin induces PP2A activity and reduces tau phosphorylation at PP2A-dependent epitopes in murine primary neurons from wild-type and human tau transgenic mice, in vitro and in vivo (Kickstein, E., et al., 2010 Proc. Natl. Acad. Sci. USA 107, 21830). These authors alleged that metformin interferes with the association of the catalytic subunit of PP2A (PP2Ac) to the so-called MID1-α4 protein complex, which regulates the degradation of PP2Ac and thereby influences PP2A activity. Kickstein et al. reported that when used at 100 nM concentration in transgenic mice, metformin decreased phosphorylation of Ser202 slightly, whereas at 10 μM, phosphorylation of Ser202 was significantly reduced. However, to achieve this level of reduction in phosphorylation of Ser202 in the brain, delivery of the drug to the CNS must be comparable to peak plasma levels using oral delivery. Further, Chen et al. reported that monotherapy with metformin, at doses that lead to activation of the adenosine monophosphate-activated protein kinase (AMPK), significantly increases the generation of both intracellular and extracellular amyloid beta species, and that the effect of metformin on amyloid beta generation is mediated by transcriptional up-regulation of beta-secretase, which results in an elevated protein level and increased enzymatic activity. Such observations suggest that monotherapy with oral metformin would be contraindicated in treatment of neurological diseases. (Chen, Y. et al., 2009 PNAS USA 106, 3907).

Craft recently reported that patients administered intranasal insulin (20 and 40 IU) had less decline in cognition compared with the placebo group on the Alzheimer Disease's Assessment Scale-cognitive subscale (Craft, S. et al., 2011 Arch. Neurol. 69(1), 29). Patients treated with intranasal insulin (20 or 40 IU) for 4 months showed an improved recall of episodic memories relative to those who were treated with placebo. However, the improved recall was not dosage dependent. Subchronic treatment with intranasal insulin, independent of the dose, preserved the patient's ability to cope with daily activities, and relatively improved general cognitive abilities, compared with the placebo group. The same patients, when tested for brain glucose utilization, exhibited a reduced decline in brain glucose uptake and utilization over the 4-month period compared with the placebo group. PET scans revealed that insulin-treated subjects showed an enhanced metabolic integrity in the precuneus region, as well as in the frontal and occipital cortices. (Schioth, H. B., et al., 2012 Expert Rev. Clin. Pharmacol. 5(1):17-20.)

However, while intranasal delivery is being investigated for delivery of some agents, control over the transport rate and effective dosage is not possible using current methods and formulations, due to negligible brain penetration.

### SUMMARY OF THE INVENTION

The present specification discloses pharmaceutical formulations for treating neurological diseases comprising a pharmaceutically active agent-transport moiety complex and a pharmaceutically acceptable carrier, wherein said formulation is suitable for administration via an intranasal route. The neurological disease is not particularly limited, and can include diverse neurological diseases and conditions associated with reduced brain insulin signaling (i.e., CNS insulin insensitivity), reduced dopaminergic signaling, reduced serotonergic signaling, reduced cholinergic signaling, or reduced GABAergic signaling, and the like. For example, the neurological diseases and conditions include Alzheimer's disease, Parkinson's disease, epilepsy, neuropathic pain, fibromyalgia, post-herpetic neuralgia, insomnia, or anxiety. The neurological diseases and conditions can include CNS cancers or conditions associated with CNS cancers.

In some embodiments, the pharmaceutically active agent is a neurotransmitter or neurotransmitter analog. For example, the pharmaceutically active agent can be gabapentin, pregabalin, atagabalin, or similar GABA analog. Another example of neurotransmitter or neurotransmitter analog is dopamine.

In some embodiments, the pharmaceutically active agent is an anti-hyperglycemic agent. Preferred anti-hyperglycemic agents comprise a guanidinium moiety. For example, the pharmaceutically active agent can be selected from metformin, phenformin, buformin, synthalin, or homologs or derivatives thereof. Such pharmaceutically active agents can be useful when intranasally administered to the CNS at a concentration that may be toxic to peripheral tissues such as liver when administered at a comparable dose systemically. In some embodiments, the formulation can contain more than one pharmaceutically active agent-transport moiety complex. For example, the formulation can comprise a dopamine-linoleate complex and a metformin-linoleate complex for treatment of conditions associated with Parkinson's disease and Alzheimer's disease, where symptoms associated with both conditions are present in a single patient.

In some embodiments, the pharmaceutically active agent-transport moiety complex is characterized by a tight ion-pair bond. For example, the pharmaceutically active agent-transport moiety complex can comprise a biguanide such as metformin and a fatty acid such as linoleic acid. Similarly, other biguanides (e.g., buformin) or other compounds containing a guanidium moiety (e.g., synthalin) can form tight ion-pair bonds. Amines such as dopamine can form tight ion-pair bonds.

In other embodiments, the pharmaceutically active agent-transport moiety complex is characterized by a loose ion-pair bond. For example, gabapentin can form loose ion-pair bonds with a fatty acid.

The transport moiety can be a fatty acid. In some embodiments, the transport moiety is a fatty acid having a branched or straight chain alkyl or alkenyl group comprising 5 to 23 carbon atoms, or mixtures thereof. Particular transport moieties can be selected from caprylic acid, lauric acid, oleic acid, linoleic acid, or arachidonic acid, or mixtures thereof. Other transport moieties such as tocopherol salts can be used to form transport moiety complexes, as described in U.S. Patent Application Publication No. 2003/0220301, incorporated by reference herein.

In some embodiments, the pharmaceutical formulation is in the form of a cream, liquid, spray, powder, or suppository. The formulation is suitable for administration via intranasal delivery. The formulation can comprise a biocompatible adhesive, for example, the formulation can be in the form of a saline suspension comprising bioadhesive particles.

Methods for treating neurological diseases and conditions are also disclosed. The methods comprise intranasally administering a pharmaceutical formulation comprising a pharmaceutically active agent-transport moiety complex. The neurological disease is associated with reduced brain insulin signaling (i.e., CNS insulin insensitivity), reduced dopaminergic signaling, reduced serotonergic signaling, reduced cholinergic signaling, reduced GABAergic signaling, or the like. The neurological disease can be Alzheimer's disease, Parkinson's disease, epilepsy, neuropathic pain, fibromyalgia, post-herpetic neuralgia, insomnia, or anxiety. The neurological diseases and conditions can include CNS cancers, metastases to the CNS, or conditions associated with CNS cancers.

In some embodiments, the methods for treating neurological diseases and conditions associated with CNS insulin insensitivity comprise intranasally administering a pharmaceutical formulation comprising an anti-hyperglycemic agent-transport moiety complex. The anti-hyperglycemic agent can comprise a guanidinium moiety, (e.g., a biguanide) and can be selected from metformin, phenformin, buformin, synthalin, or homologs or derivatives thereof. In some embodiments, the pharmaceutical formulation further comprises insulin.

The transport moiety can be a fatty acid having a branched or straight chain alkyl or alkenyl group comprising 5 to 23 carbon atoms, or mixtures thereof. Preferred transport moieties include caprylic acid, lauric acid, oleic acid, linoleic acid, arachidonic acid, or mixtures thereof.

The formulation can be in the form of a cream, liquid, spray, powder, or suppository as long as the formulation is suitable for administration via intranasal delivery. The formulation can comprise a biocompatible adhesive.

In some embodiments, the methods comprise intranasally administering the formulation of comprising the pharmaceutically active agent-transport moiety complex on a prescribed schedule.

In some embodiments, the method for treating a neurological disease comprises intranasally administering a pharmaceutical formulation comprising a dopamine-transport moiety complex. The pharmaceutical formulation can further comprise an anti-hyperglycemic agent-transport moiety complex for treatment of conditions associated with Parkinson's disease and Alzheimer's disease, where symptoms associated with both conditions are present in a single patient.

These and other embodiments and implementations are described in detail in the drawing, the description and the claims. Additional objects, advantages and novel features of the invention will be set forth in part in the description which follows, and in part will become apparent to those skilled in the art upon examination of the following, or may be learned by practice of the invention.

### DETAILED DESCRIPTION OF THE INVENTION

### I. Definitions and Overview

Before the present invention is described in detail, it is to be understood that unless otherwise indicated this invention is not limited to specific antibodies, antibody fragments, or the like, as such may vary. It is also to be understood that the terminology used herein is for the purpose of describing particular embodiments only and is not intended to limit the scope of the present invention.

It must be noted that as used herein and in the claims, the singular forms “a,” “and” and “the” include plural referents unless the context clearly dictates otherwise. Thus, for example, reference to a “transport moiety” can include one or more species of transport moiety molecules; reference to “a pharmaceutically active agent” includes two or more pharmaceutically active agents, and so forth.

Where a range of values is provided, it is understood that each intervening value, to the tenth of the unit of the lower limit unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

The term “complex” as used herein refers to a substance comprising a drug moiety Dr (e.g., metformin) and a transport moiety T (e.g., a fatty acid) associated by a tight ion-pair bond. A drug-moiety-transport moiety complex (Dr⁺T⁻) can be distinguished from a loose ion-pair of the drug moiety and the transport moiety by a difference in octanol/water partitioning behavior, characterized by the following relationship:

Δ log D=log D(complex)−log D(loose ion-pair)≧0.15 (1)

where D, the distribution coefficient (apparent partition coefficient), is the ratio of the equilibrium concentrations of all species of the drug moiety and the transport moiety in octanol to the same species in water (deionized water) at a set pH (typically about pH=5.0 to about pH=7.0) and at 25° C. Log D (complex) is determined for a complex of the drug moiety and transport moiety prepared according to the teachings herein. Log D (loose ion-pair) is determined for a physical mixture of the drug moiety and the transport moiety in deionized water. For instance, the octanol/water apparent partition coefficient (D=C_{octanol}/C_{water}) of a putative complex (in deionized water at 25° C.) can be determined and compared to a 1:1 (mol/mol) physical mixture of the transport moiety and the drug moiety in deionized water at 25° C. If the difference between the log D for the putative complex Dr⁺T⁻ and the log D for the 1:1 (mol/mol) physical mixture, Dr⁺∥T⁻ is determined to be greater than or equal to 0.15, the putative complex is confirmed as being a complex according to the invention. In some embodiments, Δ log D for the complex may be greater than or equal to 0.20, 0.25, or 0.35.

The term “tight ion-pair” refers to a pair of ions that, at physiologic pH and in an aqueous environment, are not readily interchangeable with other loosely paired or free ions that may be present in the environment of the tight ion-pair. A tight ion-pair can be experimentally detected by noting the absence of interchange of a member of a tight ion-pair with another ion, at physiologic pH and in an aqueous environment, using isotopic labeling and NMR or mass spectroscopy. Tight ion-pairs also can be found experimentally by noting the lack of separation of the ion-pair, at physiologic pH and in an aqueous environment, using reverse phase HPLC. For example, U.S. Patent Application Publication No. 2006/0094782 to Wong describes the octanol/water partitioning and HPLC elution behavior of the complex metformin laurate which forms a tight ion pair. This complex shows a LogD of 0.44 compared to the 1:1 mixture of metformin HCl: Sodium laurate which shows a LogD of 0.06.

The term “transport moiety” refers to a compound that is capable of forming a complex with a drug, wherein the transport moiety serves to improve transport of the drug across epithelial tissue, compared to that of the uncomplexed drug. The transport moiety comprises a hydrophobic portion and an acidic, basic, or zwitterionic structural element. In a preferred embodiment, the transport moiety comprises a hydrophobic portion and an acid. The hydrophobic portion can comprise a hydrocarbon chain. In an embodiment, the pKₐ of an acidic structural element is less than about 7.0, preferably less than about 6.0. In an embodiment, the pKₐ of a basic structural element is greater than about 7.0, preferably greater than about 8.0.

By “loose ion-pair” is meant a pair of ions that are, at physiologic pH and in an aqueous environment, are readily interchangeable with other loosely paired or free ions that may be present in the environment of the loose ion pair. Loose ion-pairs can be found experimentally by noting interchange of a member of a loose ion-pair with another ion, at physiologic pH and in an aqueous environment, using isotopic labeling and NMR or mass spectroscopy. Loose ion-pairs also can be found experimentally by noting separation of the ion-pair, at physiologic pH and in an aqueous environment, using reverse phase HPLC. Loose ion-pairs may also be referred to as “physical mixtures,” and are formed by physically mixing the ion-pair together in a medium. A loose ion-pair can be used effectively in nasal delivery due to the absence of counter-ions which could make the complex unstable.

The terms “level,” “amount,” and “concentration” are used as commonly understood in the art of medicine and biochemistry. These terms are used interchangeably throughout the present disclosure.

As used herein, the phrase “neurological deficits” includes an impairment or absence of a normal neurological function or presence of an abnormal neurological function.

The term “therapeutically effective amount” is used to refer to an amount of an active agent having the ability to cure disease, or ameliorate symptoms of disease. In particular aspects, the therapeutically effective amount refers to a target cerebrospinal fluid concentration that has been shown to be effective in, for example, slowing or preventing disease progression, or hastening recovery. Efficacy can be measured in conventional ways, depending on the condition to be treated. For example, in patients with Alzheimer's disease, efficacy can be measured by assessing cognitive function in patients; in patients with Parkinson's disease, efficacy can be measured by assessing control of volitional movements.

The terms “treat,” “treatment,” and “therapy” and the like are meant to include therapeutic as well as prophylactic, or suppressive measures for a disease or disorder leading to any clinically desirable or beneficial effect, including but not limited to alleviation of one or more signs or symptoms, regression, slowing or cessation of progression of the disease or disorder. Thus, for example, the term treatment includes the administration of an agent prior to or following the onset of a sign or symptom of a disease or disorder thereby preventing or removing all signs or symptoms of the disease or disorder. As another example, the term includes the administration of an agent after clinical manifestation of the disease to combat the signs and symptoms of the disease. Further, administration of an agent after onset and after clinical signs or symptoms have developed where administration affects clinical parameters of the disease or disorder, such as the degree of tissue injury or mortality, whether or not the treatment leads to amelioration of the disease, comprises “treatment” or “therapy” within the context of the invention.

By “fatty acid” is meant any of the group of organic acids of the general formula CH₃(CₙHₓ)COOH where the hydrocarbon chain is either saturated (x=2n, e.g. palmitic acid, C₁₅H₃₁COOH) or unsaturated (x=2n−2, e.g., oleic acid, CH₃C₁₆H₃₀COOH).

The term “pharmaceutically active agent” refers to a drug, compound, or agent, or a residue of such a drug, compound, or agent, that provides some pharmacological effect when administered to a subject. For use in forming a complex, the drug comprises an acidic, basic, or zwitterionic structural element, or an acidic, basic, or zwitterionic residual structural element.

The terms “cancer” or “cancers” of the central nervous system (CNS) generally refer to tumors of the brain, spinal cord, etc. such as neuroblastomas, glioblastomas, and the like. CNS cancers also include metastases to the CNS from peripheral cancers such as lung or breast cancers.

Applicant has discovered that intranasal delivery of pharmaceutically active agents can be an effective method of providing active agents for the treatment of neurological diseases such as Parkinson's disease and Alzheimer's disease as well as CNS cancers. In contrast to prior approaches to treating neurological diseases, Applicant has discovered a way to deliver pharmaceutically active agents to the brain via intracellular diffusion through the nasal epithelium whereby the passive diffusion of a drug across the nasal epithelium is enhanced when the drug is complexed to a transport moiety. The present inventor surprising discovered that otherwise impermeant or poorly permeant pharmaceutically active agents can be converted into a hydrophobic and therefore permeable form by complexation with a fatty acid, and that this complex can be delivered to the brain for the treatment of neurological diseases using intranasal delivery.

Accordingly, in one embodiment, pharmaceutical formulations comprising a pharmaceutically active agent-transport moiety complex and a pharmaceutically acceptable carrier are provided for treating neurological diseases. The formulations are suitable for administration via an intranasal route.

### II. Pharmaceutically Active Agents

The pharmaceutically active agent is effective to treat a neurological disease. The neurological disease is not particularly limited, and suitable pharmaceutically active agents are those that can be used with therapeutic effect on diverse neurological diseases and conditions associated with reduced brain insulin signaling (i.e., CNS insulin insensitivity), reduced dopaminergic signaling, reduced serotonergic signaling, reduced cholinergic signaling, or reduced GABAergic signaling, and the like. In some embodiments, pharmaceutically active agents useful for treatment of systemic diseases (outside the CNS) can also be effective to treat neurological diseases when intranasally administered using the pharmaceutically active agent-transport agent complexes of the present invention. For example, anti-hyperglycemic agents, typically systemically administered for treatment of diabetes, can be utilized in the present formulations for intranasal administration for treatment of neurological diseases and conditions.

Thus, in one embodiment, active agents are provided that obviate brain hypometabolism, e.g., agents that facilitate glucose metabolism in brain tissues. Such agents may commonly be used at anti-hyperglycemic agents for treatment of symptoms of diabetes or metabolic syndrome. Metformin is an exemplary anti-hyperglycemic agent of the biguanide class used in the treatment of type II diabetes. The structure of metformin is

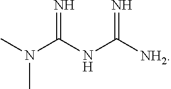

Metformin is commercially available as a hydrochloride salt, metformin HCl, and is marketed as GLUCOPHAGE for treatment of non-insulin-dependent diabetes mellitus (type II diabetes). Metformin is a cationic, water-soluble compound with a pKₐ of 12.4. At physiological pH (i.e., values less than 7.0), metformin hydrochloride is hydrophilic. The combination of hydrophilicity and charge tends to severely limit its transport across the intestinal epithelium and the blood-brain barrier via transcellular pathways. Although metformin is a small molecule, absorption of metformin from the blood to the brain in animal and humans is negligible. For patients with diabetes, a once-daily metformin treatment is desired since pharmacodynamic advantages are provided by a relatively constant dosage of metformin in the bloodstream. Accordingly, prior art approaches to metformin formulations and modes of administration have focused on sustained release formulations of metformin. Complexes for improved intestinal or colonic absorption are described in U.S. Patent Application Publication Nos. 2005/0158374 and 2006/0094782 to Wong and 2003/0220301 to Lal, each of which is incorporated by reference herein in its entirety.

In some embodiments, the active agent comprises a guanidium moiety, for example, the agent can be a substituted biguanide, including but not limited to metformin and its analogs, the related biguanides phenformin (phenethyl substituted for methyl) and buformin (butyl substituted for methyl), as well as homologs and derivatives thereof. For example, substituted biguanides (e.g., C₂-C₂₀ branched or unbranched alkyl, optionally substituted with C₅-C₁₀ aryl, halo, nitro) can also be used so long as they provide enhanced glucose metabolism in brain tissues. Biguanides linked by intervening alkyl or substituted alkyl groups, such as Synthalin, 2-[10-(diaminomethylideneamino)decyl]guanidine dihydrochloride, can also affect glucose metabolism. Although Synthalin was found to have liver toxicity limiting its use for systemic treatment, this compound can be useful in the context of CNS treatment and limited systemic dosages and liver exposure. Similarly, the lactic acidosis associated with systemic use of phenformin and other biguanides may not be a limitation in the context of CNS treatment, where lower dosages and local lactate metabolism is effective to prevent elevated levels of lactate. Additional homologs of metformin which can be used in the present methods and formulations include the biguanides described by Kim et al. in U.S. Patent Application Publication No. 2012/0309799, which is incorporated by reference in its entirety herein.

In some embodiments, the pharmaceutically active agent is a neurotransmitter or neurotransmitter analog. For example, the neurotransmitter can be γ-aminobutyric acid (GABA) and the pharmaceutically active agent can be GABA, gabapentin, pregabalin, atagabalin, or similar GABA analog. Another example of neurotransmitter or neurotransmitter analog is dopamine.

In some embodiments, a combination of active agents can be provided in the formulation. For example, the pharmaceutically active agents can be metformin and insulin (e.g., in an amount between 1 and 20 IU). In some embodiments, a combination of active agents can be provided in the formulation, for example, the pharmaceutically active agents can be metformin and one or more biguanides as described below. In some embodiments, a combination of active agents can also be provided in the formulation, for example, the pharmaceutically active agents can be metformin and dopamine as described below.

### III. Preparation of Drug-Transport Moiety Complexes

Pharmaceutically active agent-transport moiety complexes can be formed from numerous combinations of active agents and transport moieties. For example, U.S. Patent Application Publication Nos. 2005/0158374 and 2006/0094782 to Wong and 2003/0220301 to Lal, each of which is incorporated by reference herein in its entirety, describe preparation of fatty acid and tocopherol complexes of metformin.

In one embodiment, linoleic acid is an exemplary fatty acid transport moiety. It will be understood that linoleic acid is merely exemplary and that the preparation procedure can be equally applicable to other species suitable as a transport moiety, and to fatty acids of any carbon chain length. For example, complex formation of metformin with various fatty acids or salts of fatty acids, the fatty acids having from 6 to 18 carbon atoms, more preferably 8 to 16 carbon atoms and even more preferably 10 to 14 carbon atoms is contemplated. The fatty acids or their salts can be saturated or unsaturated. Exemplary saturated fatty acids contemplated for use in preparation of the complex include butanoic (butyric); pentanoic (valeric); hexanoic (caproic); octanoic (caprylic); nonanoic (pelargonic); decanoic (capric); dodecanoic (lauric); tetradecanoic (myristic); hexadecanoic (palmitic); heptadecanoic (margaric); and octadecanoic (stearic). Unsaturated fatty acids include oleic acid, linoleic acid, and linolenic acid, all having 18 carbon atoms. Linoleic acid and linolenic acid are polyunsaturated. Other unsaturated fatty acids can also be used as desired.

Complex formation of active agents with alkyl sulfates or a salt of an alkyl sulfate is also contemplated, where the alkyl sulfate may be saturated or unsaturated. Exemplary alkyl sulfates, or their salts (sodium, potassium, magnesium, etc), have from 6 to 18 carbon atoms, more preferably 8 to 16 and even more preferably 10 to 14 carbon atoms. Complex formation of active agents with the benzenesulfonic acid, benzoic acid, fumaric acid, and salicylic acid, or the salts of these acids, is also contemplated.

In one embodiment, complexes in accord with the invention can include a complex of metformin-thiocitic acid (also known as alpha-lipoic acid).

Preparation of pharmaceutically active agent-transport moiety complexes can be prepared by any convenient methods known in the art. Example 1 illustrates a typical method for the preparation of metformin linoleate. Briefly, the method includes preparing the basic form of the active agent, mixing the active agent with a transport moiety (e.g., a lipophilic acid) in a solvent (typically nonaqueous), forming the complex by allowing sufficient time and providing favorable temperature and solvent conditions, collecting the complex (e.g., a precipitate), and optionally purifying the product (e.g., washing the precipitate). A similar process can be employed to prepare a formulation comprising dopamine linoleate.

### IV. Formulations

Formulations comprising a pharmaceutically active agent-transport moiety provided in accordance with embodiments of the invention can be used as medicaments (or in the preparation of a medicament) intended for the treatment and/or prevention of a wide range of neurological diseases and disorders. The rationale for intranasal administration of drugs is that the drugs may be retained in the submucous space of the nose, cross the arachnoid membrane, and enter into the central nervous system via the olfactory pathways. Without being bound by theory, it is believed that the use of a transport moiety complex facilitates transport of the pharmaceutically active agent to the CNS, thereby improving response time and minimizing exposure of peripheral tissues to active agents.

However, there is no clear equivalence between the once daily oral dose of a particular drug and the effective CNS dose of the same drug when delivered intranasally. The pharmacodynamics of intranasal delivery to CNS targets is completely different from the pharmacodynamics of systemic (e.g., oral or parenteral) delivery to peripheral targets. The present inventor surprisingly discovered that intranasal delivery of the instant pharmaceutically active agent-transport moiety complex can provide a daily dose of from about 10 μg to about 50 mg of drug-transport moiety complex to be absorbed and delivered to the CNS. Daily dosing can be performed as frequently as every hour (the nostrils are cleared by mucociliary action in 20 to 40 minutes). Dosing from 1 to 6 times per day is preferable. Formulations can be provided at any convenient concentration, such as 10 to 30% (w/v) drug in aqueous saline solutions.

Accordingly, in some embodiments, the formulations provide a dose of metformin-transport moiety complex that is from about 10 μg to about 50 mg of metformin-transport moiety complex to be absorbed and delivered to the CNS. In some embodiments, the formulations provide a dose of metformin-transport moiety complex that is between about 100 μg and about 10 mg. In some embodiments, the formulations provide a dose of metformin-transport moiety complex that is between about 1 mg and about 5 mg. To increase the contact time and targeting to the olfactory nerves, formulation of a pharmaceutically active agent-transport moiety with a biocompatible adhesive or a delivery device can be prepared. The formulation is not particularly limiting, and can be in the form of a cream, liquid, spray, powder, or suppository which can be administered intranasally using a suitable applicator. Processes for preparing pharmaceuticals in these vehicles can be found throughout the literature.

In some embodiments, the pharmaceutically active agent-transport moiety complex is soluble, and the formulation is a solution. In some embodiments, the pharmaceutically active agent-transport moiety complex is insoluble, and the formulation provides the pharmaceutically active agent-transport moiety complex as a suspension of nanoparticles, or a micro-emulsion. The formulation can be applied using any convenient method or device such as a spray device, metered dose applicator for cream, suppository suitable for intranasal insertion, and the like.

The compositions described herein can also comprise a bioadhesive agent, for example, a mucoadhesive agent. The mucoadhesive agent permits a close and extended contact of the composition, or the drug released from said composition, with mucosal surface by promoting adherence of said composition or drug to the mucosa. The mucoadhesive agent is preferably a polymeric compound, such as preferably, a cellulose derivative but it may be also a natural gum, alginate, pectin, or such similar polymer. The most preferred cellulose derivative is hydroxypropyl methylcellulose available under the trade name METHOCEL®, commercially available from Dow Chemical Co. The mucoadhesive agent can be present in from about 5 to about 25%, by weight, preferably in from about 10 to about 15% and most preferably about 10%.

Bioadhesive microparticles or nanoparticles can constitute still another component of the intranasal formulations suitable for use in the present invention. The bioadhesive particles include derivatives of cellulose such as hydroxypropyl cellulose and polyacrylic acid and can provide sustained release of the pharmaceutically active agents for an extended period of time (possibly days) once they are placed in the appropriate formulation. A formulation comprising bioadhesive particles can provide a multi-phase liquid or semi-solid preparation which does not seep from the nose. The microparticles or nanoparticles cling to the nasal epithelium and can release the drug over extended period of time, for example, for several hours or more. Many of these bioadhesive systems are described in U.S. Pat. Nos. 4,756,907, and 6,200,590, incorporated herein by reference. The bioadhesive formulation can comprise microparticles or nanoparticles in addition to the pharmaceutically active agent-transport moiety complex, and may optionally contain a surfactant for enhancing solubility and/or uptake of the drug, so long as the surfactant does not disrupt the pharmaceutically active agent-transport moiety complex. The microparticles typically have a diameter of 1-100 μm, whereas nanoparticles typically have a diameter of 10-1000 nm. Microparticles and nanoparticles can be prepared from starch, gelatin, albumin, collagen, or dextran according to methods known in the art.

The biocompatible adhesives can include viscosity enhancers such as methylcellulose, sodium carboxymethylcellulose, chitosan, carbopol 934P and Pluronic 127. Thermogelling agents such as ethyl(hydroxyethyl) cellulose and Pluronic 127 can also be used to advantage. Thermogelling agents are liquid at room temperature and below, but at physiological temperatures (e.g., 32-37° C.), the viscosity of the solution increases such that the solution becomes a gel.

Pharmaceutical compositions may be formulated in combination with any suitable pharmaceutical vehicle, excipient or carrier that would commonly be used in this art, such as saline, dextrose, water, glycerol, ethanol, other therapeutic compounds, and combinations thereof. As one skilled in this art would recognize, the particular vehicle, excipient or carrier used will vary depending on the patient and the patient's condition, and a variety of modes of administration would be suitable for the compositions of the invention, as would be recognized by one of ordinary skill in this art.

Suitable nontoxic pharmaceutically acceptable excipients for use in the compositions of the present invention will be apparent to those skilled in the art of pharmaceutical formulations and examples are described in REMINGTON: The Science and Practice of Pharmacy, 20^{th} Edition, A. R. Gennaro, ed., (2000). The choice of suitable carriers will depend on the exact nature of the particular vaginal dosage form desired, e.g., whether the chemotherapeutic agent and/or inhibitor of membrane efflux systems is/are to be formulated into a cream, lotion, foam, ointment, paste, solution, microemulsions, liposomal suspension, microparticles, nanoparticles or gel, as well as on the physicochemical properties of the active ingredient(s).

### V. Methods for Treating Neurological Diseases

Methods for treating neurological diseases and conditions are also disclosed that comprise intranasally administering to a subject in need thereof a therapeutically effective amount of a pharmaceutically active agent-transport moiety complex as disclosed herein. Neurological disease and conditions include central nervous system disorders in a subject, e.g., a human subject. Examples of central nervous system disorders include, but are not limited to: a myelopathy, an encephalopathy, central nervous system (CNS) infection, encephalitis (e.g., viral encephalitis, bacterial encephalitis, parasitic encephalitis), meningitis (e.g., spinal meningitis, bacterial meningitis, viral meningitis, fungal meningitis), neurodegenerative diseases (e.g., Huntington's disease, Alzheimer's disease, Parkinson's disease, multiple sclerosis, amyotrophic lateral sclerosis, traumatic brain injury), mental health disorder (e.g., schizophrenia, depression, dementia), pain and addiction disorders, brain tumors (e.g., intra-axial tumors, extra-axial tumors), adult brain tumors (e.g., glioma, glioblastoma), pediatric brain tumors (e.g., medulloblastoma), cognitive impairment, genetic disorders (e.g., Huntington's disease, neurofibromatosis type 1, neurofibromatosis type 2, Tay-Sachs disease, tuberous sclerosis), headache (e.g., tension headache, migraine headache, cluster headache, meningitis headache, cerebral aneurysm and subarachnoid hemorrhage headache, brain tumor headache), stroke (e.g., cerebral ischemia or cerebral infarction, transient ischemic attack, hemorrhagic (e.g., aneurysmal subarachnoid hemorrhage, hypertensive hemorrhage, other sudden hemorrhage)), epilepsy; spinal disease (e.g., degenerative spinal disease (e.g., herniated disc disease, spinal stenosis, and spinal instability), traumatic spine disease, spinal cord trauma, spinal tumors, hydrocephalus (e.g., communicating or non-obstructive hydrocephalus, non-communicating or obstructive hydrocephalus, adult hydrocephalus, pediatric hydrocephalus, normal pressure hydrocephalus, aqueductal stenosis, tumor associated hydrocephalus, pseudotumor cerebri), CNS vasculitis (e.g., primary angiitis of the central nervous system, benign angiopathy of the central nervous system, Arnold Chiari malformation), neuroAIDS, retinal disorders (e.g., age-related macular degeneration, wet age-related macular degeneration, myopic macular degeneration, retinitis pigmentosa, proliferative retinopathies), inner ear disorders, tropical spastic paraparesis, arachnoid cysts, locked-in syndrome, Tourette's syndrome, adhesive arachnoiditis, altered consciousness, autonomic neuropathy, benign essential tremor, brain anomalies, cauda equine syndrome with neurogenic bladder, cerebral edema, cerebral spasticity, cerebral vascular disorder, and Guillain-Barre syndrome.

The methods can be used to treat neurological deficits due to neurodegeneration in the brain of a subject. The method can include intranasally administering a pharmaceutically active agent-transport moiety complex as described herein to the subject. Neurodegeneration of the brain can be the result of disease, injury, and/or aging. As used herein, neurodegeneration includes morphological and/or functional abnormality of a neural cell or a population of neural cells. Non-limiting examples of morphological and functional abnormalities include physical deterioration and/or death of neural cells, abnormal growth patterns of neural cells, abnormalities in the physical connection between neural cells, under- or over production of a substance or substances, e.g., a neurotransmitter, by neural cells, failure of neural cells to produce a substance or substances which it normally produces, production of substances, e.g., neurotransmitters, and/or transmission of electrical impulses in abnormal patterns or at abnormal times. Neurodegeneration can occur in any area of the brain of a subject and is seen with many disorders including, for example, head trauma, stroke, Amylotrophic lateral sclerosis, multiple sclerosis, Huntington's disease, Parkinson's disease, and Alzheimer's disease.

The methods comprise intranasally administering a pharmaceutical formulation comprising a pharmaceutically active agent-transport moiety complex. A dose of pharmaceutically active agent can be provided that is from about 10 μg to about 50 mg of pharmaceutically active agent-transport moiety complex to be absorbed and delivered to the CNS. In some embodiments, the neurological disease is associated with reduced brain insulin signaling (i.e., CNS insulin insensitivity), reduced dopaminergic signaling, reduced serotonergic signaling, reduced cholinergic signaling, reduced GABAergic signaling, or the like. The neurological disease can be Alzheimer's disease, Parkinson's disease, epilepsy, neuropathic pain, fibromyalgia, post-herpetic neuralgia, insomnia, or anxiety.

The present inventor has surprisingly recognized that intranasal administration of an anti-hyperglycemic agent such as a biguanide (e.g., metformin or phenformin, etc.) can provide therapeutic efficacy to conditions of brain hypometabolism (impaired insulin signaling due to CNS insulin resistance). Intranasal delivery of the formulations described herein can provide enhanced insulin sensitivity and insulin signaling, and thereby provide effective treatment and/or prevention of cognitive deficits such as Alzheimer's disease and can provide an effective treatment for CNS cancers. The use of a pharmaceutically active agent-transport moiety complex can provide enhanced CNS delivery and stability and targeted dosing control.

In some embodiments, the methods for treating neurological diseases and conditions associated with CNS insulin insensitivity comprise intranasally administering a pharmaceutical formulation comprising an anti-hyperglycemic agent-transport moiety complex. The anti-hyperglycemic agent can comprise a guanidinium moiety, (e.g., a biguanide) and can be selected from metformin, phenformin, buformin, synthalin, or homologs or derivatives thereof. In some embodiments, a dose of metformin is provided that is from about 10 μg to about 50 mg of metformin-transport moiety complex to be absorbed and delivered to the CNS. Additional active agents can also be included in the formulation. In some embodiments, the pharmaceutical formulation further comprises insulin, in an amount between 1 and 20 IU.

In some embodiments, the methods comprise intranasally administering the formulation of comprising the pharmaceutically active agent-transport moiety complex on a prescribed schedule.

Methods for treating neurological diseases associated with reduced dopaminergic signaling are provided. The methods comprise intranasally administering a formulation comprising a dopamine-transport moiety complex. Most diseases and conditions associated with reduced dopamine signaling are treated by oral administration of L-dopa because dopamine is not able to cross the BBB from the systemic circulation. The methods of the present invention allow the use of a formulation comprising a dopamine-transport moiety complex to treat conditions by intranasal administration. Because intranasal administration bypasses the BBB, dopamine can be administered in its active form and not as a precursor. In some embodiments, a dose of dopamine is provided that is from about 10 μg to about 50 mg of dopamine-transport moiety complex to be absorbed and delivered to the CNS.

Neurological diseases associated with reduced dopaminergic signaling may be reflected by movement anomalies or motor disorders. Typically, these diseases and conditions are associated with Parkinson's disease, but can include other disorders such as a dyskinesia, postencephalitic parkinsonism, dopa-sensitive dystonia, Shy-Drager syndrome, periodic limb movement disorder (PLMD) syndrome, periodic limb movements in sleep (PLMS) syndrome, Tourette's syndrome or restless legs syndrome (RLS), and the like.

Parkinson's disease and Alzheimer's disease can occur at the same time in certain patients. The present methods also include the use of a formulation comprising a dopamine-transport moiety complex (e.g., a dopamine-linoleate complex) and an anti-hyperglycemic agent-transport moiety complex (e.g., a metformin-linoleate complex) for treatment of conditions associated with Parkinson's disease and Alzheimer's disease, where symptoms associated with both conditions are present in a single patient.

Methods for treating neurological diseases associated with reduced GABAergic signaling are provided. The methods comprise intranasally administering a formulation comprising a GABA or GABA analog-transport moiety complex. The pharmaceutically active agent can be GABA, gabapentin, pregabalin, atagabalin, or similar GABA analog. Exemplary transport moieties for complex formation with GABA or GABA analogs include alkyl sulfates.

Neurological diseases associated with reduced GABAergic signaling typically include epilepsy, neuropathic pain, fibromyalgia, post-herpetic neuralgia, insomnia, or anxiety, but can include other disorders that can be treated with GABA or GABA analogs.

### VI. Methods for Treating CNS Cancers

Methods for treating and/or preventing CNS cancers and malignancies are also disclosed. Anti-hyperglycemic agents that activate adenosine monophosphate-activated protein kinase (AMPK) can be useful in treating cancer, and using the novel formulations and methods disclosed herein, can be useful for treating and preventing brain cancers. For example, Buzzai et al. reported that when metformin is used to treat p53 gene-deficient colon cancer cells, metformin activates AMPK of the cancer cells and affects the metabolic energy pathway, thereby killing the cancer cells because they cannot adjust to the changed metabolic pathway (Buzzai, M. et al. 2007 Cancer Res 67: (14)]). Ferla et al. reported that metformin reduced basal and leptin-stimulated growth of glioblastoma cells in a dose-dependent manner, and that it also significantly inhibited the migration of glioblastoma cells. They also reported that the action of metformin was mediated through the upregulation of its main signaling molecule, AMPK, as well as through the downregulation of the signal transducer and activator of transcription 3 (STAT3) and the Akt/PKB serine/threonine protein kinase (Ferla, R. et al. 2012 Oncol Lett. 5, 1077-1081). There is further evidence that metformin can reduce or eliminate cancer stem cells (Song et al. 2012 Sci Rep. 2, 362).

Methods for treating and/or preventing CNS cancers and malignancies include intranasally administering an anti-hyperglycemic agent-transport moiety complex. The anti-hyperglycemic agent can comprise a guanidinium moiety, (e.g., a biguanide) and can be selected from metformin, phenformin, buformin, synthalin, or homologs or derivatives thereof. Additional biguanides which can be used in the present methods and formulations include the biguanides described by Kim et al. in U.S. Patent Application Publication No. 2012/0309799, which is incorporated by reference in its entirety herein. In some embodiments, a dose of the antihyperglycemic agent (e.g., a biguanide such as metformin) is provided that is from about 10 μg to about 50 mg of biguanide-transport moiety complex (e.g., metformin-transport moiety complex) to be absorbed and delivered to the CNS. Additional active agents can also be included in the formulation. In some embodiments, the pharmaceutical formulation further comprises insulin, in an amount between 1 and 20 IU. Additional anticancer agents can be included in the formulation.

The methods comprise intranasally administering a pharmaceutical formulation comprising a pharmaceutically active agent-transport moiety complex. A dose of pharmaceutically active agent can be provided that is from about 50 mg to about 250 mg of anti-hyperglycemic agent-transport moiety complex. The present inventor has surprisingly recognized that intranasal administration of an anti-hyperglycemic agent such as a biguanide (e.g., metformin or phenformin) in the form of an active agent-transport moiety complex can provide therapeutic efficacy to treating or preventing cancerous conditions in the CNS. The use of an anti-hyperglycemic agent-transport moiety complex can provide enhanced delivery and stability and targeted dosing control.

In some embodiments, the methods for treating cancer comprise intranasal administration of a pharmaceutical formulation comprising an anti-hyperglycemic agent-transport moiety complex. The anti-hyperglycemic agent can comprise a guanidinium moiety, (e.g., a biguanide) and can be selected from metformin, phenformin, buformin, synthalin, or homologs or derivatives thereof. In some embodiments, a dose of metformin is provided that is from about 10 μg to about 50 mg of metformin-transport moiety complex. Additional active agents can also be included in the formulation. In some embodiments, the pharmaceutical formulation further comprises insulin, in an amount between 1 and 20 IU.

In some embodiments, the methods comprise intranasally administering the formulation comprising the pharmaceutically active agent-transport moiety complex on a prescribed schedule, such as once per day, two times per day, three times per day, four times per day, and so forth.

CNS and brain cancers that can be treated using the intranasal delivery of the present active-agent-transport moiety complex include, but are not limited to, brain and other central nervous system tumors (e.g. tumors of the meninges, brain, spinal cord, cranial nerves and other parts of central nervous system): glioma, glioblastoma, acoustic neuroma pilocytic astrocytoma, oligodendroglioma, astrocytoma, meningioma, anaplastic astrocytoma, lymphoma, vestibular schwannoma, multiforme adenoma, medulloblastoma, metastatic brain tumor, craniopharyngioma, meningioma, neuroblastoma ependymoma, spinal tumor, pinealoma, medulloblastoma, hemangioblastoma. Glial tumors, the most prevalent and morbid of which are high-grade astrocytoma and the aggressive glioblastoma multiforme, are the most common brain tumors in adults. They are also among the least treatable cancers, with a 3 year survival after initial diagnosis of <10% for tumors initially diagnosed at the grade 4 (glioblastoma) stages. The current treatments of glioma and glioblastoma achieve only palliation and short-term increments in survival. They include surgical resection, following which ultimate recurrence rates are over 90%, as well as radiation therapy, and chemotherapies.

Neurologic cancers include childhood brain stem childhood cerebellar astrocytoma, childhood cerebral astrocytoma/malignant glioma, childhood ependymoma, childhood medulloblastoma, childhood pineal and supratentorial primitive neuroectodermal tumors, childhood visual pathway and hypothalamic gliomas, other childhood brain cancers, adrenocortical carcinoma; central nervous system lymphoma, primary, neuroblastoma, craniopharyngioma, spinal cord tumors, central nervous system atypical teratoid/rhabdoid tumor, central nervous system embryonal tumors, and childhood supratentorial primitive neuroectodermal tumors and pituitary tumor, pineocytoma, pineoblastoma, and primary brain lymphoma. Neurological cancers also include brain metastases of cancers of non-CNS origin such as breast and lung cancer.

In certain embodiments, a pharmaceutically active agent-transport moiety complex described herein may be administered alone or in combination with other compounds useful for treating or preventing a systemic metabolic disorder, e.g., diabetes or metabolic syndrome. Exemplary agents that can be co-administered systemically in conjunction with the intranasal delivery of the pharmaceutically active agent-transport moiety complexes include, for example, alpha-glucosidase inhibitors such as miglitol (GLYSET®), acarbose (PRECOSE®); amylin analogs such as pramlintide (SYMLIN®); dipeptidyl peptidase 4 inhibitors such as sitagliptin (JANUVIA®), saxagliptin (ONGLYZA®), tolbutamide (ORINASE®), linagliptin (TRADJENTA®); insulin such as insulin glulisine (APIDRA®, APIDRA SOLOSTAR®), insulin glargine (LANTUS®, LANTUS SOLOSTAR®), insulin lispro (HUMALOG®, HUMALOG KWIKPEN®), insulin zinc (HUMULIN L®, HUMULIN U®, ILETIN LENTE®, LENTE ILETIN II®, NOVOLIN L®), insulin detemir (LEVEMIR®), insulin aspart (NOVOLOG®), insulin isophane (HUMULIN N®, HUMULIN N Pen®, NOVOLIN N®, RELION NOVOLIN N®), insulin (EXUBERA®, HUMULIN R®, NOVOLIN R®, RELION/NOVOLIN R®, VELOSULIN BR10); incretin mimetics such as exenatide (BYDUREON®, BYETTA®), liraglutide (VICTOZA®); meglitinides such as repaglinide (PRANDIN®), nateglinide (STARLIX®), sulfonylureas such as glimepiride (AMARYL®), glyburide (DIABETA®, GLYCRON®, GLYNASE®, GLYNASE PRESTAB®, MICRONASE®), chlorpropamide (DIABINESE®), acetohexamide (DYMELOR®), glipizide (GLIPIZIDE XL®, GLUCOTROL®, GLUCOTROL XL®), tolbutamide (TOL-TAB®, TOLINASE®); non-sulfonylureas such as metformin (FORTAMET®, GLUCOPHAGE®, GLUCOPHAGE XR®, GLUMETZA®, RIOMET®); thiazolidinediones such as pioglitazone (ACTOS®), rosiglitazone (AVANDIA®), troglitazone (REZULIN®), minerals and electrolytes such as chromium picolinate (Cr-GTF®, CRM®); and antidiabetic combinations such as metformin/pioglitazone (ACTOPLUS Met®, ACTOPLUS MET XR®); metformin/rosiglitazone (AVANDAMET®, AVANDARYL®), metformin/saxagliptin (KOMBIGLYZE XR®), glimepiride/pioglitazone (DUETACT®), glyburide/metformin (GLUCOVANCE®), metformin/sitagliptin (Janumet®), simvastatin/sitagliptin (JUVISYNC®), glipizide/metformin (METAGLIP®), metformin/repaglinide (PRANDIMET®).

In some embodiments, the methods include co-administration with systemic delivery of an anti-cancer agent such as temozolamide, bevacizumab, procarbazine, lomustine, vincristine, gefitinib, erlotinib, docetaxel, cis-platin, 5-fluorouracil, gemcitabine, tegafur, raltitrexed, methotrexate, cytosine arabinoside, hydroxyurea, adriamycin, bleomycin, doxorubicin, daunomycin, epirubicin, idarubicin, mitomycin-C, dactinomycin and mithramycin, vinblastine, vindesine, vinorelbine, taxol, taxotere, etoposide, teniposide, amsacrine, topotecan, camptothecin, bortezomib, anagrelide, tamoxifen, toremifene, raloxifene, droloxifene, iodoxyfene, fulvestrant, bicalutamide, flutamide, nilutamide, cyproterone, goserelin, leuprorelin, buserelin, megestrol, anastrozole, letrozole, vorazole, exemestane, finasteride, marimastat, trastuzumab, cetuximab, dasatinib, imatinib, combretastatin, thalidomide, and/or lenalidomide or combinations thereof.

### VII. Modes of Administration

Intranasal delivery of pharmaceutically active agent-transport moiety complexes can be provided by a variety of different means, and will vary depending upon the intended application. In some embodiments, the pharmaceutically active agent-transport moiety complex is provided in a formulation for application to the nasal epithelium. The formulation can be in the form of a cream, liquid, spray, powder, or suppository. A metered dose of the formulation can be provided from a reservoir of the formulation. In addition, predetermined dosages can be provided, for example, suppository forms can be provided for insertion into the nose having a predetermined dosage. Kits can be provided, where prepared dosage forms and instructions for administering the dosages are included.

In some embodiments, the pharmaceutically active agent-transport moiety complexes are delivered using a ViaNase™ nasal drug delivery device (Kurve Technology, Inc., Lynnwood, Wash.). This device can release a metered dose of pharmaceutically active agent-transport moiety complex into a chamber covering the participant's nose, which is then inhaled by breathing regularly for a sufficient time (e.g., 2 minutes) to allow the prescribed dose to be delivered. For example, each spray can deliver a volume of approximately 250 microliter, or ˜250 mg. For a 20 to 30% (w/v) solid load, each administration will contain a maximum of about 50 mg of drug-transport moiety complex. A titration process can be used to determine the optimum dosage per day: the number of spays to arrive at a desired target penetration (dose delivered) can be determined empirically by measuring the effect on neurological function or using other biochemical marker or assay.

An additional delivery method utilizes a delivery device to deliver solid drug particles suspended in a viscous gel such as petrolatum on the olfactory mucosa of the patient's nostril. In some embodiments, a delivery device includes a soft hollowed tube with an injector mechanism for delivering the suspended drug particles to the desired location.

In one embodiment, the invention provides a nasal spray device for intranasal delivery of the pharmaceutically active agent-transport moiety complexes. For example, the OptiNose liquid delivery nasal spray device and other similar devices that deliver drug to both the olfactory and respiratory epithelium of the nasal cavity, while preventing delivery to the lungs by spraying only when the connection between the nose and lungs is closed, may be employed to deliver the pharmaceutically active agent-transport moiety complexes. In one embodiment, the nasal spray device is a pressurized olfactory delivery device such as the Impel NeuroPharma device, a metered dose nasal spray device or a unit dose nasal spray device. In one embodiment, the device provides for delivery of the pharmaceutically active agent-transport moiety complexes high in the nasal cavity so as to reach the olfactory epithelium, thereby maximizing delivery to the brain.

In addition, devices for facilitating the intranasal delivery include those described in U.S. Patent Application Publication No. 20110120456 to Immel, which is incorporated by reference herein. Devices to deliver the intranasal formulation are not particularly limited, and the devices discussed herein are only exemplary.

The formulations can be administered on a prescribed schedule. The formulations can be administered as a bolus or over a period of time in a controlled delivery fashion, for example, as a continuous infusion (e.g., at a constant rate of delivery or at a varying rate) or in a pulsatile fashion.

### VIII. Assessment of Treatment Efficacy

Efficacy of treatment can be assessed using any convenient methods. For example, the Alzheimer Disease's Assessment Scale-cognitive subscale can be utilized to measure changes in cognitive function in Alzheimer's disease or dementia patients (Rosen W. G., et al. 1984 Am. J. Psychiatry 141, 1356-1364). Similarly, other assessment criteria are known in the art for determining whether symptoms such as dyskinesias, pain, seizures, anxiety, and the like are ameliorated by a given dosage and formulation. Biochemical and diagnostic assays can also be performed in order to assess whether a patient shows improvement in particular neurological functions or diagnostic parameters. For example, PET scanning can be performed to assess functional activity in the brain using ¹⁸F-deoxyglucose or the like. In addition, the PET amyloid imaging agent ¹⁸F-florbetapir can be used to assess amyloid-beta pathology in patients, or its progression or regression.

Similarly, PET scanning can be performed to assess the glucose uptake activity of CNS tissues as well as the size and glucose uptake activity of CNS tumors. Cerebrospinal fluid can be sampled and subjected to biochemical analysis. For example, Teplyuk, N. M. et al. reported that the levels of miR-10b and miR-21 have been found significantly increased in the CSF of patients with glioblastoma and brain metastasis of breast and lung cancer, compared with tumors in remission and a variety of normeoplastic conditions. Members of the miR-200 family are highly elevated in the CSF of patients with brain metastases but not with any other pathologic conditions, allowing discrimination between glioblastoma and metastatic brain tumors. (Teplyuk, N. M. et al. 2012 Neuro. Oncol. 14, 689). In addition, CSF markers for Alzheimer's Disease can be measured. These markers include β-amyloid1-42 (A(β1-42), total tau protein (T-tau) and hyperphosphorylated tau (P-tau181P). Similar markers known in the art for Parkinson's Disease and other neurological diseases mentioned herein can be utilized to evaluate treatment efficacy.

It is to be understood that while the invention has been described in conjunction with the preferred specific embodiments thereof, that the description above as well as the examples that follow are intended to illustrate and not limit the scope of the invention. The practice of the present invention will employ, unless otherwise indicated, conventional techniques of organic chemistry, pharmaceutical chemistry, immunochemistry, biochemistry and the like, which are within the skill of the art. Other aspects, advantages and modifications within the scope of the invention will be apparent to those skilled in the art to which the invention pertains. Such techniques are explained fully in the literature.

All patents, patent applications, and publications mentioned herein, both supra and infra, are hereby incorporated by reference.

### Example 1

### Preparation of an Intranasal Formulation of Metformin-Transporter Moiety Complex

### Preparation of Metformin Base

A procedure similar to that disclosed in U.S. Patent Application Publication No. 2005/0158374 for the preparation of metformin laurate is performed. An ion exchange column is packed with 200 g of the anionic resin, Amberlyst A-26 (OH) and a net weight is obtained. The column is rinsed first with deionized (DI) water (backflushed) and then rinsed with methanol containing 2% v/v DI water, with care taken to not allow the column to dry out.

Metformin hydrochloride (29.48 g) is dissolved in an eluant comprised of 650 mL methanol containing 2% DI water by volume. The metformin solution is passed through the column dropwise using a separatory funnel and the eluate is collected. The total metformin hydrochloride passed through is calculated to be less than the ion exchange resin's equilibrating point (capacity). The column is rinsed with 200 mL methanol containing 2% (v/v) DI water. The eluate of the metformin base is collected. The combined eluates are evaporated to dryness under vacuo at an external temperature of 40° C., and raised to 65° C. at the end of the concentration step to remove all the remaining water. The metformin sample is dried in an oven overnight at room temperature.

### Complex Formation

Acetone (500 mL) is added to the metformin base with stirring at 500 rpm for 15 min. The metformin HCl is filtered out using a 0.45 micron filter under vacuum, and the filtrate is collected (any unreacted metformin HCl remains on the filter). Linoleic acid (50 g) is dissolved in 460 mL acetone. From a separatory funnel, the linoleic acid-acetone solution is added to the metformin base solution at a fast drip. The precipitate forms and a white cloudy solution appears (metformin linoleate). After all the linoleic acid-acetone solution is added, the solution is covered and stirred for 1.5 hr at 500 rpm. In this step, pure nitrogen gas is passed through the suspension to minimize oxidation of the linoleate.

The precipitated metformin linoleate is filtered through a 0.45 micron filter on a Buchner funnel with vacuum. The precipitate is rinsed with 4×60 mL acetone. Drying under vacuum is continued for 30 min. The product is further dried in a vacuum oven at room temperature overnight. Metformin linoleate is obtained as a sticky white powder.

### Example 2

### Clinical Testing for Patients Suffering from Mild Cognitive Impairment or Alzheimer's Disease

Patients are administered a formulation comprising metformin-linoleate prepared according to Example 1 via intranasal administration of an aqueous formulation, and testing is performed to see if the preparation improves memory in adults with mild cognitive impairment (MCI) or Alzheimer's disease. Primary outcome measures include changes in cognition, changes in glucose metabolism, changes in plasma biological markers, changes in CSF markers, changes in cerebral glucose metabolism. Escalating dosages of metformin-linoleate are tested and compared with a placebo for 4 months to see if the formulation facilitates memory for adults with AD and MCI. A subset of participants will participate in two sub-studies utilizing PET scans (prior to and at the end of treatment) to determine whether intranasal metformin-linoleate increases cerebral glucose metabolism, and lumbar punctures (LPs) before and at the end of treatment to determine effects of intranasal metformin-linoleate administration on CSF Aβ levels. Study participants should be age 55 or older, be diagnosed with MCI or Alzheimer's disease.

Subjects are randomly assigned to receive a daily dosage of metformin-linoleate (i.e., participants receive metformin-linoleate twice a day), or placebo (i.e., participants receive saline twice a day) for 4 months. The dose of metformin provided is equivalent to 1/10 or 1/100 the oral dose of metformin for the subject's body weight. In other words, for an equivalent oral dose of metformin of 500 mg, the intranasal dose of metformin-linoleate is either 31.3 mg or 313 mg.

Participants are stratified by whether or not they are carriers of the ApoE ε4 allele. Saline or metformin-linoleate (prepared as described in Example 2) is administered after breakfast and dinner with a ViaNase nasal drug delivery device (Kurve Technology, Inc., Lynnwood, Wash.) designed to deliver drugs to the olfactory region to maximize transport to the central nervous system. This device releases a metered dose of metformin-linoleate into a chamber covering the participant's nose, which is then inhaled by breathing regularly for 2 minutes until the prescribed dose is delivered.

Parallel versions of the cognitive and functional protocol are administered at baseline, months 2 and 4 of treatment, and 2 months after treatment. Testing is performed in the morning after a standard meal. Participants are instructed to skip their morning dose on the day of testing and thus have received their last dose more than 12 hours prior to cognitive testing. The co-primary outcome measures are the delayed story recall score and the Dementia Severity Rating Scale (DSRS) score. The protocol consists of the following measures: (1) The delayed story recall score is determined after a story containing 44 informational bits read a single time to participants who are then asked to recall the story immediately and after a 20-minute delay. (2) The DSRS score is determined after a questionnaire is completed by the study partner; this questionnaire is used to rate the change in the participant's cognitive, social, and functional status over a specified period of time, with higher scores indicating greater impairment. (3) The Alzheimer Disease's Assessment Scale-cognitive subscale (ADAS-cog) is performed, and includes measures of memory, praxis, orientation, and language, with higher scores indicating greater impairment. (4) The Alzheimer's Disease Cooperative Study-activities of daily living (ADCS-ADL) scale is completed by the study partner and used to rate the participant's ability to perform daily activities within the past month, with lower scores indicating greater impairment.

### Lumbar Puncture

After a 12-hour fast, an intravenous catheter is inserted, and the L4-5 interspace was infiltrated with lidocaine, 1%, for anesthesia. Using a 24-gauge Sprotte spinal needle (B. Braun Medical, Bethlehem, Pa.), 30 mL of CSF is withdrawn into sterile syringes, aliquotted into prechilled polyethylene tubes, frozen immediately on dry ice, and stored at −70° C. until testing.

### Biomarkers

Levels of Aβ1-42, tau protein, and P181-tau in CSF are measured with the multiparameter bead-based immunoassay (INNO-BIA AlzBio3; Innogenetics NV, Gent, Belgium). The CSF Aβ40 level is measured by sandwich enzyme-linked immunosorbent assays using 6E10-coated plates (Signet Laboratories, Dedham, Mass.) in conjunction with biotinylated anti-Aβ40. The limit of detection is expected to be about 15 pg/mL.

Positron Emission Tomographic Imaging with Fluorodeoxyglucose

Positron emission tomographic imaging is obtained using a GE Advance PET scanner (GE Medical Systems, Milwaukee, Wis.). The participants are kept in a quiet, dimly lit room with their eyes open. Following an injection of 10 mCi of FDG, participants are monitored for 35 min, after which they were then moved to the scanner table, and a transmission scan and a brief emission scan are obtained in 2-dimensional data acquisition mode for 25 and 5 min, respectively, for post-injection attenuation correction. The final emission scan is obtained for 15 min in 3-dimensional data acquisition mode. Attenuation-corrected transaxial image sets were reconstructed using a filter-back projection method. Reconstructed attenuation-corrected 3-dimensional emission images yield an in-plane spatial resolution of approximately 5 mm.

### Example 3

### Clinical Testing for Patients Suffering from CNS Cancers

Study participants should be diagnosed with a CNS cancer such as glioblastoma. Patients are administered a formulation comprising metformin-linoleate prepared according to Example 1 via intranasal administration of an aqueous formulation, and testing is performed to see how the treatment affects tumor glucose uptake and metabolism. Primary outcome measures include changes in cognition and other neurological parameters, changes in glucose metabolism, changes in plasma tumor markers, changes in CSF markers, changes in cerebral glucose metabolism. Escalating dosages of metformin-linoleate are tested and compared with a placebo for 4 months to see if the formulation reduces tumor size in patients diagnosed with CNS cancers, e.g., glioblastoma. A subset of participants will participate in two sub-studies utilizing PET scans (prior to and at the end of treatment) to determine whether intranasal metformin-linoleate increases cerebral glucose metabolism, and lumbar punctures (LPs) before and at the end of treatment to determine effects of intranasal metformin-linoleate administration on CSF markers. The levels of miR-10b and miR-21 have been found significantly increased in the CSF of patients with glioblastoma and brain metastasis of breast and lung cancer, compared with tumors in remission and a variety of normeoplastic conditions (Teplyuk, N. M. et al. 2012 Neuro. Oncol. 14, 689). Improvements in the levels of miR-10b indicate the efficacy of the treatment.

What is claimed is:

1. A pharmaceutical formulation for treating neurological diseases comprising a pharmaceutically active agent-transport moiety complex and a pharmaceutically acceptable carrier, wherein said formulation is suitable for administration via an intranasal route.

2. The formulation of claim 1, wherein the pharmaceutically active agent is a neurotransmitter or neurotransmitter analog.

3. The formulation of claim 2, wherein the pharmaceutically active agent is gabapentin, pregabalin, or atagabalin.

4. The formulation of claim 2, wherein the pharmaceutically active agent is dopamine.

5. The formulation of claim 1, wherein the pharmaceutically active agent is an anti-hyperglycemic agent.

6. The formulation of claim 5, wherein the pharmaceutically active agent comprises a guanidinium moiety.

7. The formulation of claim 6, wherein the pharmaceutically active agent is selected from metformin, phenformin, buformin, or homologs or derivatives thereof.

8. The formulation of claim 1, wherein the transport moiety is a fatty acid.

9. The formulation of claim 8, wherein the transport moiety is a fatty acid having a branched or straight chain alkyl or alkenyl group comprising 5 to 23 carbon atoms, or mixtures thereof.

10. The formulation of claim 8, wherein the transport moiety is selected from caprylic acid, lauric acid, oleic acid, linoleic acid, or arachidonic acid, or mixtures thereof.

11. The formulation of claim 1, wherein the formulation is in the form of a cream, liquid, spray, powder, or suppository.

12. The formulation of claim 11, wherein the formulation comprises a biocompatible adhesive.

13. The formulation of claim 1, wherein the pharmaceutically active agent-transport moiety complex is characterized by a tight ion-pair bond.

14. The formulation of claim 1, wherein the pharmaceutically active agent-transport moiety complex is characterized by a loose ion-pair bond.

15. A method for treating a neurological disease comprising intranasally administering a pharmaceutical formulation comprising a pharmaceutically active agent-transport moiety complex.

16. The method of claim 15, wherein the neurological disease is associated with reduced brain insulin signaling, reduced dopaminergic signaling, reduced serotonergic signaling, reduced cholinergic signaling, reduced GABAergic signaling.

17. The method of claim 15, wherein the pharmaceutically active agent is an anti-hyperglycemic agent.

18. The method of claim 15, wherein the pharmaceutically active agent comprises a guanidinium moiety.

19. The method of claim 18, wherein the pharmaceutically active agent is selected from metformin, phenformin, buformin, or homologs or derivatives thereof.

20. The method of claim 15, wherein the neurological disease is Alzheimer's disease, Parkinson's disease, epilepsy, neuropathic pain, fibromyalgia, post-herpetic neuralgia, insomnia, or anxiety.

21. The method of claim 1, further comprising administering the formulation of claim 1 on a prescribed schedule.

22. The method of claim 15, wherein the pharmaceutically active agent is gabapentin.

23. A method for treating a neurological disease comprising intranasally administering a pharmaceutical formulation comprising a dopamine-transport moiety complex.

24. The method of claim 23, wherein the pharmaceutical formulation further comprises an anti-hyperglycemic agent-transport moiety complex.
